(19) United States
(12) Patent Application Publication
(10) Pub. No.: US 2025/0325623 A1
(43) Pub. Date: Oct. 23, 2025

(54) Process for Preparing Efficient Liver-Protecting Compound Ingredient

(71) Applicant: Andall Biosciences Inc., Torrance, CA (US)

(72) Inventor: Heju Li, Torrance, CA (US)

(21) Appl. No.: 18/735,985

(22) Filed: Jun. 6, 2024

(30) Foreign Application Priority Data
Apr. 17, 2024 (CN) 202410462992.3

(51) Int. Cl. A61K 36/8888; A61K 9/06; A61K 36/232; A61K 36/233; A61K 36/284; A61K 36/315; A61K 36/344; A61K 36/355; A61K 36/481; A61K 36/537; A61K 36/638; A61K 36/65; A61K 36/734; A61K 36/748; A61K 36/752; A61K 36/79; A61K 36/815; A61K 36/888; A61K 36/8945; A61K 36/8969; A61K 36/9066

(52) U.S. Cl. CPC A61K 36/8888; A61K 9/06; A61K 36/232; A61K 36/233; A61K 36/284; A61K 36/315; A61K 36/344; A61K 36/355; A61K 36/481; A61K 36/537; A61K 36/638; A61K 36/65; A61K 36/734; A61K 36/748; A61K 36/752; A61K 36/79; A61K 36/815; A61K 36/888; A61K 36/8945; A61K 36/8969; A61K 36/9066; A61K 2236/15; A61K 2236/333; A61K 2236/39; A61K 2236/51; A61K 2236/53

(57) ABSTRACT

The present disclosure relates to the technical field of liver-protecting ingredient preparation processes, and particularly to a process for preparing an efficient liver-protecting compound ingredient. The process comprises an internal drug and an external drug, wherein the internal drug comprises 65 g of astragalus membranaceus, 65 g of Salvia miltiorrhiza, 50 g of Ligustrum lucidum, 50 g of the fruit of Chinese wolfberry, 50 g of Tuckahoe, 40 g of radix bupleuri, 40 g of Schisandra chinensis, 40 g of Codonopsis pilosula, 40 g of Pinellia ternata, 40 g of rhizoma dioscoreae, 40 g of radix isatidis, 40 g of Atractylodes macrocephala, 40 g of radix curcumae, 40 g of Radix Paeoniae Alba, 40 g of Hedyotis diffusa, 40 g of Angelica sinensis, 40 g of pericarpium citri reticulatae, 40 g of honeysuckle, 40 g of rhizoma polygonati, and 30 g of parched hawthorn fruit.

### CROSS REFERENCE TO RELATED APPLICATION

This application claims the benefit of and takes priority from Chinese Patent Application No. 202410462992.3 filed on Apr. 17, 2024, the contents of which are herein incorporated by reference.

### TECHNICAL FIELD

The present disclosure relates to the technical field of liver-protecting ingredient preparation processes, and particularly to a process for preparing an efficient liver-protecting compound ingredient.

### BACKGROUND

A liver oral liquid is a traditional Chinese medicine preparation for liver healthcare and adjuvant therapy. With the acceleration of modern life pace and the intensification of environmental pollution, liver diseases are increasing day by day, and more and more attentions to liver health are also gradually gained by people. Therefore, the market demand for liver-protecting oral liquid is gradually increased.

At present, there are a huge usage amount of liver-protecting tablets, liver-protecting agents and other products on the market, but their liver-protecting principles are often to promote a metabolism level. However, improving the metabolism level often allows liver-protecting drugs sold on the market to slowly take effects, and cannot effectively inhibit the protection effect of the human body on liver, so as to increase the burdens of organs such as liver. To this regard, a process for preparing an efficient liver-protecting compound ingredient is provided in order to solve the above problem.

### SUMMARY

The objective of the present disclosure is to provide a process for preparing an efficient liver-protecting compound ingredient in order to solve the problem proposed in the above background.

The technical solution of the present disclosure is as follows: provided is a process for preparing an efficient liver-protecting compound ingredient, comprising an internal drug and an external drug.

The internal drug comprises 65 g of astragalus membranaceus, 65 g of Salvia miltiorrhiza, 50 g of Ligustrum lucidum, 50 g of the fruit of Chinese wolfberry, 50 g of Tuckahoe, 40 g of radix bupleuri, 40 g of Schisandra chinensis, 40 g of Codonopsis pilosula, 40 g of Pinellia ternate, 40 g of rhizoma dioscoreae, 40 g of radix isatidis, 40 g of Atractylodes macrocephala, 40 g of radix curcumae, 40 g of Radix Paeoniae Alba, 40 g of Hedyotis diffusa, 40 g of Angelica sinensis, 40 g of pericarpium citri reticulatae, 40 g of honeysuckle, 40 g of rhizoma polygonati, and 30 g of parched hawthorn fruit;
the thermal reflux extraction process of the internal drug comprises the following steps:
Step S1: smashing 65 g of Salvia miltiorrhiza, 40 g of Schisandra chinensis and 50 g of Ligustrum lucidum into crude particles, and extracting the crude particles twice by thermal reflux extraction such as ethanol thermal reflux extraction;
(1) for the first time, adding 600 ml of ethanol into the crude particles for 3 h of reflux extraction to obtain a mixed solution; for the second time, adding 450 ml of ethanol into the mixed solution for 2 h of reflux extraction, and filtering to obtain filtrate;
(2) decompressing the filtrate to recover ethanol to obtain extract;
Step S2: simultaneously placing 40 g of radix bupleuri, 40 g of Atractylodes macrocephala, 40 g of radix curcumae, 40 g of Angelica sinensis and 40 g of pericarpium citri reticulatae into a distillation vessel, adding 600 ml into the distillation vessel, and then distilling to collect distillate;
Step S3: soaking dregs obtained after reflux extraction of 65 g of astragalus membranaceus, 50 g of the fruit of Chinese wolfberry, 50 g of Tuckahoe, 40 g of Codonopsis pilosula, 40 g of Pinellia ternate, 40 g of rhizoma dioscoreae, 40 g of radix isatidis, 40 g of Radix Paeoniae Alba, 40 g of Hedyotis diffusa, 40 g of honeysuckle, 40 g of rhizoma polygonati and 30 g of parched hawthorn fruit with ethanol and dregs obtained after extraction of distillate together with water for 1 h, and performing heating and extraction twice;
(1) for the first time, adding 5000 ml of water and decocting for 2 h to obtain a mixed solution; for the second time, adding 4000 ml of water and decocting for 1.5 h, and filtering to obtain a solution; and
(2) performing vacuum concentration on the solution and then taking out the solution, adding a 5% 101 juice clarifying agent into the above solution, evenly stirring, standing and refrigerating for 24 h, and then performing vacuum concentration again to an appropriate amount to obtain a concentrated solution;
Step S4: adding the standby extract, the distillate and the concentrated solution, evenly stirring, adjusting a total volume to 1000 ml, packaging, sealing and sterilizing;
the external drug comprises 40 g of sedum sarmentosum, 40 g of barbed skullcap, 50 g of Hedyotis diffus, 10 g of Polygonum cuspidatum, 20 g of Fructus Aurantii, 30 g of Fructus Gardeniae, 40 g of oriental wormwood, 60 g of Schisandra chinensis and plaster cloth;
the decoction and extraction process of the external drug comprises the following steps:
Step S5: adding 1000 ml of water into 40 g of Sedum sarmentosum, 40 g of barbed skullcap, 50 g of Hedyotis diffusa, 10 g of Polygonum cuspidatum, 20 g of Fructus Aurantii, 30 g of Fructus Gardeniae, 40 g of oriental wormwood and 60 g of Schisandra chinensis to be soaked for 2 h;
Step S6: decocting the soaked medicinal materials for 15 min over fire to obtain a concentrated medicinal juice, and then filtering the concentrated medicinal juice to remove residues;
Step S7: pouring the filtered medicinal juice into a pot to be concentrated, decocting over high heat to accelerate the evaporation of water, and skimming off the foam at any time to gradually thicken the medicinal juice, then further concentrating over soft fire while continuously stirring until the medicinal juice does not scatter on paper because the medicinal juice is prone to sticking to the bottom, and stopping decocting at this moment to obtain a concentrated clear ointment;
Step S8: when the ointment is pasted, evenly smearing the clear ointment on plaster cloth, leaving a certain gap at the edge to facilitate the sealing of the edge, gently compacting the plaster cloth after pasting is completed to tightly bond the ointment with the plaster cloth.

Preferably, in the step S3, an instrument used for vacuum concentration is a rotary evaporator.

Preferably, the plaster cloth is cut into a size of 80 mm*80 mm, and the plaster cloth is made of a non-woven fabric material.

Preferably, in the step S1, the concentration of ethanol is 80%, and the extraction temperature is 70° C.-75° C.

Preferably, in the step S2, the distillation is short-range distillation, with a process pressure range of 1-1×10⁻³ mba and a temperature range of 150-280° C.

Preferably, in the step S3, the ratio of vacuum concentration is 1:1.5, and an addition ratio of the 5% 101 fruit juice clarifying agent to the filtrate is 1:9.

Preferably, in the step S6, filtration is performed by using a ceramic membrane filter, and the ceramic membrane filter is washed with a pH0-14 strong acid/alkaline oxidizing reagent.

Preferably, in the step S1, the mesh size of the filter screen used for filtration is 1.20 mm, and the filter screen is made of a stainless steel material.

Through improvement, the present disclosure provides a process for efficiently preparing the liver-protecting compound ingredient herein, which has the following improvements and advantages compared with the prior art:

1. In the present disclosure, lipid peroxidation is related to liver fibroplasia, which can cause liver fibrosis by affecting collagen metabolism. This formula has an anti-lipid peroxidation effect, which is one of the important mechanisms for treatment of chronic hepatitis B, reduction of liver cell damage, and liver fibrosis resistance.

2. In the present disclosure, the formula is a pure traditional Chinese medicine compound preparation, with more effective ingredients, the medicinal materials such as astragalus membranaceus, radix bupleuri and pericarpium citri reticulatae are identified by thin layer chromatography, and the content of astragaloside iv is measured by dual-wavelength thin layer chromatography scanning. The process of the present disclosure is simple, convenient, correct, high in sensitivity, good in repeatability, and can effectively control the quality of the formula and ensures clinical effects.

3. In the present disclosure, through the anti-lipid peroxidation effect of the internal drug, the liver is protected to alleviate stem cell damage and withstand liver fibrosis, and then the digestion of the fat and the nourishing of the kidney are achieved through the external drug, and the combined use of the internal drug and the external drug can effectively protect the liver.

### BRIEF DESCRIPTION OF THE DRAWINGS

Next, the present disclosure will be further explained in combination with drawings and embodiments.

FIG. 1 is a diagram showing an astragalus membranaceus thin-layer identification spectrum according to the present disclosure;

FIG. 2 is a diagram showing a radix bupleuri thin-layer identification spectrum according to the present disclosure;

FIG. 3 is a diagram showing a pericarpium citri reticulatae thin-layer identification spectrum according to the present disclosure;

FIG. 4 is a diagram showing a Radix Paeoniae Alba thin-layer identification spectrum according to the present disclosure;

FIG. 5 is a diagram showing a Codonopsis pilosula thin-layer identification spectrum according to the present disclosure;

FIG. 6 is a diagram of an Angelica sinensis thin-layer identification spectrum according to the present disclosure;

FIG. 7 is a diagram showing determination results of astragaloside iv according to the present disclosure;

FIG. 8 is a diagram showing sampling recovery rate test results according to the present disclosure;

FIG. 9 is a comprehensive curative effect comparison diagram of an observation group and a control group according to the present disclosure.

### DETAILED DESCRIPTION OF THE EMBODIMENTS

Next, the present disclosure will be described in detail. The technical solution of the present disclosure will be clearly and completely described, obviously, the described embodiments are only some embodiments of the present disclosure, but not all the embodiments. Based on the embodiments of the present disclosure, other embodiments obtained by persons of ordinary skill in the art without creative efforts are all included within the scope of protection of the present disclosure.

Through improvement, the present disclosure provides a process for preparing an efficient liver-protecting compound ingredient. The technical solution of the present disclosure is as follows:

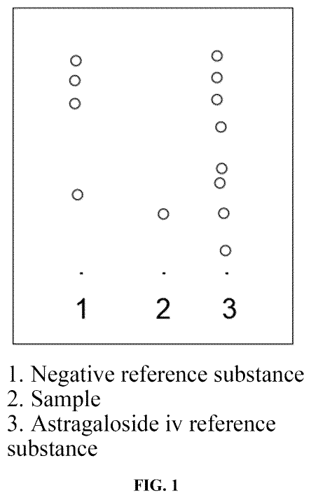

FIG. 1-FIG. 9 show a process for preparing an efficient liver-protecting compound ingredient. Next, the present disclosure will be described in detail. The technical solution in the embodiments of the present disclosure will be clearly and completely described, obviously, the described embodiments are only some embodiments of the present disclosure, but not all the embodiments. Based on the embodiments of the present disclosure, other embodiments obtained by persons of ordinary skill in the art without creative efforts are all included within the scope of protection of the present disclosure.

Through improvement, the present disclosure provides a process for preparing an efficient liver-protecting compound ingredient. The technical solution of the present disclosure is as follows:
a process for preparing an efficient liver-protecting compound ingredient comprises an internal drug and an external drug;
wherein the internal drug comprises 65 g of astragalus membranaceus, 65 g of Salvia miltiorrhiza, 50 g of Ligustrum lucidum, 50 g of the fruit of Chinese wolfberry, 50 g of Tuckahoe, 40 g of radix bupleuri, 40 g of Schisandra chinensis, 40 g of Codonopsis pilosula, 40 g of Pinellia ternate, 40 g of rhizoma dioscoreae, 40 g of radix isatidis, 40 g of Atractylodes macrocephala, 40 g of radix curcumae, 40 g of Radix Paeoniae Alba, 40 g of Hedyotis diffusa, 40 g of Angelica sinensis, 40 g of pericarpium citri reticulatae, 40 g of honeysuckle, 40 g of rhizoma polygonati, and 30 g of parched hawthorn fruit.

Identification of astragalus membranaceus: 30 g of astragalus membranaceus was taken and extracted twice (20 ml and 20 ml) with diethyl ether, a diethyl ether solution was discarded, the astragalus membranaceus was extracted 3 times (30 ml, 20 ml, and 20 ml) with water saturated n-butanol, extracting solutions were merged, the merged filtrate was washed 3 times (20 ml, 20 ml, and 15 ml) with a n-butanol saturated 1% sodium hydroxide solution, the alkaline solution was discarded, the remaining solution was washed with n-butanol saturated water until being neutral, the aqueous solution was discarded, the n-butanol solution was evaporated to dryness in a water bath, the residue was dissolved by adding 1 ml of methanol as a test sample solution; astragaloside iv was used as a reference substance, and a 1 mg/ml solution was prepared by adding methanol as a reference substance solution. 4 μl of each of the above two solutions was sucked and applied to the same silica gel G thin layer plate separately, developed by using a lower layer of chloroform-ethyl acetate-methanol-water (10:20:11:5) placed below 10° C. as a developing agent, and taken out, dried in air, sprayed with a 10% sulfuric acid ethanol solution, and heated for about 5 min at 105° C. The results show that in the test sample spectrum, the same tan spots appear at a place corresponding to a reference substance spectrum; in the UV-lamp (a wavelength of 365 nm) view, the same orange spots appear. The negative control test proves that there is no interference, see FIG. 1.

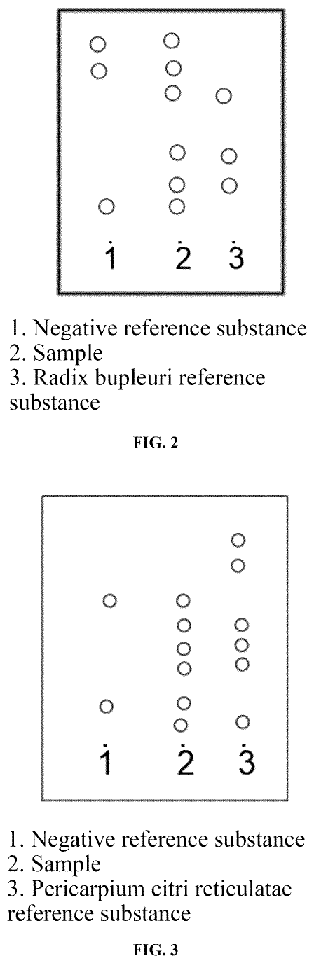

Identification of radix bupleuri: 1.5 g of radix bupleuri was taken, 4 μl of each of a radix bupleuri reference medicinal material solution and an astragalus membranaceus test sample solution were sucked and applied to the same silica gel G-CMC thin layer plate separately, developed by using a lower-layer solution of chloroform-ethyl acetate-methanol-water (7:3:1) as a developing agent, taken out, dried in air, sprayed with a 10% sulfuric acid ethanol solution of 5% p-dimethylaminobenzaldehyde, and heated for a few minutes at 105° C. The results show that in the test sample spectrum, two identical pink spots appear at a place corresponding to the control medicinal material spectrum. The negative control test proves that there is no interference, see FIG. 2.

Identification of pericarpium citri reticulatae: 40 g of pericarpium citri reticulatae was taken and extracted three times (30 ml, 20 ml, and 20 ml) with ethyl acetate, extracting solutions were merged and then evaporated to dryness in a water bath, the residues were dissolved by adding 1 ml of absolute ethanol as a test sample solution; 2 g of pericarpium citri reticulatae control medicinal material powder was taken and appropriately decocted for 30 min with water, then the decocted solution was filtered, the filtrate and the test sample solution were prepared into a reference medicinal material solution; 4 μl of test sample solution and 2 μl of reference medicinal material solution were taken and applied to the same silica gel G plate prepared by a OH solution in a preparation process of 1% N efficient liver-protecting compound ingredient separately, upward developed by 3 cm with an upper layer solution of ethyl acetate-methanol-water (100:17:13), taken out and then downward developed by 8 cm with the upper layer solution of toluene-ethyl acetate-water (20:10:1:1) as a developing agent, and subsequently taken out, and sprayed with a 3% aluminum trichloride ethanol solution. The above two solutions were placed in the UV-lamp (a wavelength of 365 nm) view. The results show that the same blue white fluorescent spots appear in the middle of the spectrum at a place corresponding to the reference medicinal material. The negative control test proves that there is no interference, see FIG. 3.

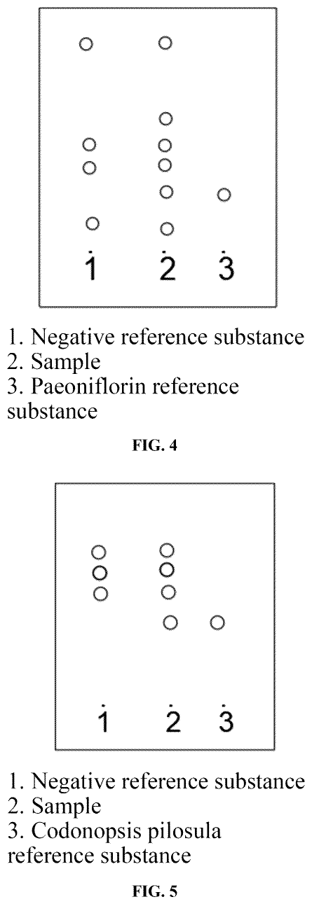

Identification of Radix Paeoniae Alba: 30 g of Radix Paeoniae Alba was taken and extracted twice (15 ml and 15 ml) with diethyl ether, a diethyl ether layer was discarded, a water layer was extracted three times with water saturated n-butyl alcohol, with 20 ml each time. The water layer was then washed with n-butyl alcohol saturated water, with 20 ml each time. The aqueous solution was discarded, the n-butyl alcohol solution was evaporated to dryness, and the residue was dissolved by adding 1 ml of methanol as a test sample solution. The Radix Paeoniae Alba reference substance was taken, and prepared into a 1 mg/ml solution as a reference substance solution by adding methanol. 3 μl of test sample solution and 2 μl of reference substance solution were sucked and then applied to the same silica gel G-CMC thin layer plate separately, developed by using chloroform-ethyl acetate-methanol-formic acid (40:5:10:0 2) as a developing agent, taken out, dried in air, sprayed with a 5% vanillin sulfuric acid solution, and then heated until spots shows clear colors. The results show that in the test sample spectrum, blue purple spots appear at a place corresponding to the reference substance spectrum. The negative control test proves that there is no interference, see FIG. 4.

Identification of Codonopsis pilosula: 30 g of Codonopsis pilosula was taken and extracted twice by adding diethyl ether, with 20 ml each time. The diethyl ether solution was discarded, and the remaining extraction solution was extracted three times with n-butanol, with 20 ml each time. The extraction solutions were merged, the merged extraction solution was evaporated to dryness in a water bath, and the residue was dissolved by adding 1 ml of ethanol as a test sample solution. 1 g of Codonopsis pilosula was taken and appropriately decocted for 30 min by adding water, the decocted medicinal material solution was filtered, and the filtrate was prepared into a reference medicinal material solution according to the preparation method of the test sample solution. 2 μl of each of the two solutions was sucked and applied to a silica gel G thin layer plate separately, developed by using n-butanol-ethanol-water (15:3:2) as a developing agent, then taken out, dried in air, sprayed with a 10% sulfuric acid ethanol solution, and dried at 105° C. until the color is clear. The results show that in the test sample spectrum, the same taupe spots appear at a place corresponding to the control medicinal material spectrum. The negative control test proves that there is no interference, see FIG. 5.

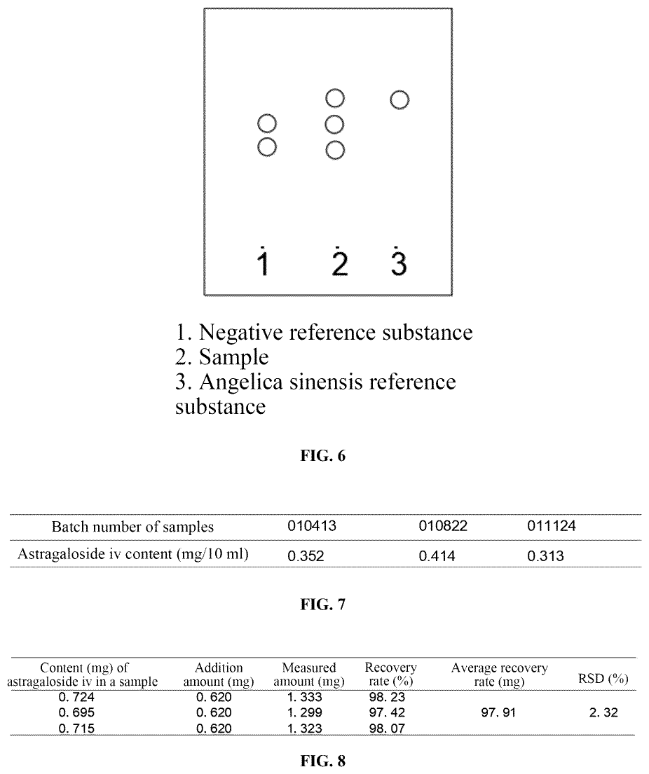

Identification of Angelica sinensis: 20 g of Angelica sinensis was taken, the pH was adjusted to 2-3 with dilute hydrochloric acid, and the Angelica sinensis solution was extracted three times with ethyl acetate, with 20 ml each time. The extraction solutions were merged, the merged extraction solution was dehydrated with anhydrous sodium sulfate and filtered, the filtrate was evaporated to be dryness, and the residue was dissolved by adding 1 ml of ethanol as a test sample solution. Ferulic acid was taken as a reference substance, and prepared into 1 mg/ml solution by adding ethanol as a reference substance solution. 3 μl of each of the two solutions was taken and applied to a silica gel G thin layer plate separately, developed by using benzene-chloroform-glacial acetic acid (30:25:5), taken out, dried in air, sprayed with 1% ferric chloride and 1% potassium ferrocyanide aqueous solutions (equal amounts were mixed when in use). The results show that in the test sample spectrum, the same color sports appear at a place corresponding to control spectrum. The negative control test proves that there is no interference, see FIG. 6.

### Content Measurement of Astragaloside iv

Quantitative conditions for chromatography: an adsorbent was a silica gel G thin layer plate (10 cm×20 cm, 0.5 mm in thickness); a developing agent: a lower-layer solution of chloroform-ethyl acetate-methanol-water (10:20:11:5) below 10° C.; a color developer: a 10% sulfuric acid ethanol solution, dry for about 5 min at 105° C.

Scanning conditions: a dual-wavelength reflection method was selected for zigzag scanning. λ S=530 nm, λ R=700 nm, slit 1.2 mm×1.2 mm, SX=3; sensitivity: medium.

Linear relationship examination: a 1 mg/ml of astragaloside iv reference substance was weighed and 1, 2, 3, 4, and 5 μl of astragaloside iv reference substances were applied to the same thin layer plate, developed according to experimental conditions, colored and measured by scanning, and then their peak areas were calculated. The results show that the content of astragaloside iv is between 1 μg and 5 μg, exhibiting a good linear relationship. The regression equation: Y=1584.32X+823.42, r=0.9994 (n=3).

Determination of a sample: 30 ml of sample was precisely weighed, the residue was dissolved by adding 5 ml of water, cooled, eluted with 100 ml of water in a D101 macroporous adsorption resin column (with an inner diameter of 1.5 cm, and a length of 14 cm) and then eluted with 50 ml of 30% ethanol, the eluent was discarded, the remaining solution was eluted with 80ml of 70% ethanol, the elutent was collected and evaporated to dryness, the residue was dissolved by adding methanol in batch, and the dissolved residue was transferred into a 2 ml of measuring bottle. 4 μl of test sample solution and 4 μl of reference substance solution were precisely sucked and then applied to the same thin layer plate separately and then developed in turn. After coloring, scanning measurement was performed. The results are seen in FIG. 7.

Sampling and recovery rate test: a sample with a known content was precisely weighed, a certain amount of astragaloside iv reference substance was precisely added into the weighed sample. Measurement was performed according to the method, and the recovery rate was calculated. The results are seen in FIG. 8.

### Sample Stability Test

3 batches of samples were stored at 37° C.-40° C. under a relative humidity of 75%, with inspection once per 1 mo for continuous 3 mo in total. The results show that the character, identification, relative dense, pH value, hygienic examination and content measurement of the sample all meet the specification;

the thermal reflux extraction process of the internal drug comprises the following steps:
Step S1: smashing 65 g of Salvia miltiorrhiza, 40 g of Schisandra chinensis and 50 g of Ligustrum lucidum into crude particles, and extracting the crude particles twice by thermal reflux extraction such as ethanol thermal reflux extraction;
(1) for the first time, adding 600 ml of ethanol into the crude particles for 3 h of reflux extraction to obtain a mixed solution; for the second time, adding 450 ml of ethanol into the mixed solution for 2 h of reflux extraction, and filtering to obtain filtrate;
(2) decompressing the filtrate to recover ethanol to obtain extract;
Step S2: simultaneously placing 40 g of radix bupleuri, 40 g of Atractylodes macrocephala, 40 g of radix curcumae, 40 g of Angelica sinensis and 40 g of pericarpium citri reticulatae into a distillation vessel, adding 600 ml into the distillation vessel, and then distilling to collect distillate;
Step S3: soaking dregs obtained after reflux extraction of 65 g of astragalus membranaceus, 50 g of the fruit of Chinese wolfberry, 50 g of Tuckahoe, 40 g of Codonopsis pilosula, 40 g of Pinellia ternate, 40 g of rhizoma dioscoreae, 40 g of radix isatidis, 40 g of Radix Paeoniae Alba, 40 g of Hedyotis diffusa, 40 g of honeysuckle, 40 g of rhizoma polygonati and 30 g of parched hawthorn fruit with ethanol and dregs obtained after extraction of distillate for 1 h by adding water, and performing heating and extraction twice;
(1) for the first time, adding 5000 ml of water and decocting for 2 h to obtain a mixed solution; for the second time, adding 4000 ml of water and decocting for 1.5 h, and filtering to obtain a solution;
(2) performing vacuum concentration on the solution and then taking out the solution, adding a 5% 101 juice clarifying agent into the above solution, evenly stirring, standing and refrigerating for 24 h, and then performing vacuum concentration again to an appropriate amount to obtain a concentrated solution;
Step S4: adding the standby extract, the distillate, the concentrated solution and an additive, evenly stirring, adjusting a total volume to 1000 ml, packaging, sealing and sterilizing.

The external drug comprises 40 g of sedum sarmentosum, 40 g of barbed skullcap, 50 g of Hedyotis diffus, 10 g of Polygonum cuspidatum, 20 g of Fructus Aurantii, 30 g of Fructus Gardeniae, 40 g of oriental wormwood, 60 g of Schisandra chinensis and plaster cloth;
the decoction and extraction process of the external drug comprises the following steps:
Step S5: adding 1000 ml of water into 40 g of Sedum sarmentosum, 40 g of barbed skullcap, 50 g of Hedyotis diffusa, 10 g of Polygonum cuspidatum, 20 g of Fructus Aurantii, 30 g of Fructus Gardeniae, 40 g of oriental wormwood and 60 g of Schisandra chinensis to be soaked for 2 h;
Step S6: decocting the soaked medicinal materials for 15 min over fire to obtain a concentrated medicinal juice, and then filtering the concentrated medicinal juice to remove residues;
Step S7: pouring the filtered medicinal juice into a pot to be concentrated, decocting over high heat to accelerate the evaporation of water, and skimming off the foam at any time to gradually thicken the medicinal juice, then further concentrating over soft fire while continuously stirring until the medicinal juice does not scatter on paper because the medicinal juice is prone to sticking to the bottom, and stopping decocting at this moment to obtain a concentrated clear ointment;
Step S8: when the ointment is pasted, evenly smearing the clear ointment on plaster cloth, leaving a certain gap at the edge to facilitate the sealing of the edge, and then gently compacting the plaster cloth after pasting is completed to tightly bond the ointment with the plaster cloth.

### Clinical Control Application

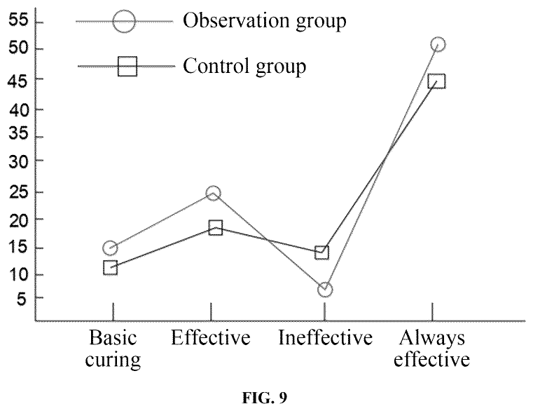

118 patients with chronic hepatitis B were randomly divided into an observation group and a control group, wherein there were 59 patients in observation group, including 36 males and 23 females, with an average age of (34.12±12.68) a; there were 59 patients in control group, including 36 males and 23 females, with an average age of (34.12±12.68) a. The patients in the observation group took 50 ml of the product each time twice a day and used liver-protecting patches once every 24 hours; the patients in the control group received intravenous infusion of 100 ml of Qiangli Ning and intramuscular injection of 15 mg of thymosin, once a day. The patients in the two groups received 3 mo as a course of treatment. By judgment according to the Internal Medicine Liver Disease Professional Committee of the Chinese Society of Traditional Chinese Medicine formulated the “Traditional Chinese Medicine Efficacy Evaluation Standards for Viral Hepatitis (Trial)” in 1992, via statistical analysis, the curative effect of the observation group was obviously superior to that of the control group (P<0.05), see FIG. 9.

Further, in the step S3, an instrument used for vacuum concentration was a rotary evaporator. By using the rotary evaporator, the evaporation efficiency can be improved, the operation temperature was reduced, thereby effectively improving the yield of the product, separating the product and recovering useful substances.

Further, the plaster cloth was cut into a size of 80 mm*80 mm, and the plaster cloth was made of a non-woven fabric material. Cutting the plaster cloth into the size of 80 mm*80 mm helps ensure the fit and comfort of the plaster. The plaster cloth was made of a non-woven fabric material, which makes the plaster cloth have good air permeability, moisture absorption and durability. The non-woven fabric material is soft, comfort, friendly to skin, and suitable for long-term use.

Further, in the step S1, the concentration of ethanol was 80%, and the extraction temperature was 70° C.-75° C. The extraction temperature of 70° C.-75° C. is a higher temperature, but cannot cause the boiling of ethanol, can accelerate the dissolution of ethanol on a target ingredient and meanwhile cannot damage the structure or activity of the target ingredient. In addition, the temperature can also reduce the pollution and propagation of microorganisms.

Further, the step S2, the distillation was short-range distillation, with a process pressure range of 1-1×10⁻³ mba and a temperature range of 150-280° C. The short-range distillation can achieve the effect of efficiently purifying, maintaining active ingredients, saving energy and reducing consumption, improving the quality of the product, etc.

Further, in the step S3, the ratio of vacuum concentration was 1:1.5, and an addition ratio of the 5% 101 fruit juice clarifying agent to the filtrate was 1:9. The treatment process also facilitates the removal of undesirable components in the filtrate, such as suspended particles and impurities, thereby improving the purity of the filtrate.

Further, in the step S6, the filtration was performed by using a ceramic membrane filter, and the ceramic membrane filter was washed with a pH0-14 strong acid/alkaline oxidizing reagent. First, when the concentrated medicinal juice was filtered through a ceramic membrane filter, the residues and impurities in the medicinal juice would be intercepted by the membrane, while clear filtrate would flow out through the membrane.

Further, in the step S1, the mesh size of the filter screen used for filtration was 1.20 mm, and the filter screen was made of a stainless steel material. Due to high strength and durability, the stainless steel material can bear large pressure and friction force, and the filter screen is not easy to damage and can maintain stable filtration effect under the high-load working conditions, thereby improving the working efficiency.

By the above descriptions, those skilled in the art can implement or use the present disclosure. Various modifications to these embodiments are obvious for those skilled in the art. The general principle defined herein can be implemented in other embodiments without departing from the spirit or scope of the present disclosure. Therefore, the present disclosure will not be limited to these embodiments of the present disclosure, but will conform to the widest range consistent with the principles and novel features disclosed herein.

What is claimed is:

1. A process for preparing an efficient liver-protecting compound ingredient, comprising an internal drug and an external drug,
wherein the internal drug comprises 65 g of astragalus membranaceus, 65 g of Salvia miltiorrhiza, 50 g of Ligustrum lucidum, 50 g of the fruit of Chinese wolfberry, 50 g of Tuckahoe, 40 g of radix bupleuri, 40 g of Schisandra chinensis, 40 g of Codonopsis pilosula, 40 g of Pinellia ternate, 40 g of rhizoma dioscoreae, 40 g of radix isatidis, 40 g of Atractylodes macrocephala, 40 g of radix curcumae, 40 g of Radix Paeoniae Alba, 40 g of Hedyotis diffusa, 40 g of Angelica sinensis, 40 g of pericarpium citri reticulatae, 40 g of honeysuckle, 40 g of rhizoma polygonati, and 30 g of parched hawthorn fruit;
the thermal reflux extraction process of the internal drug comprises the following steps:
Step S1: smashing 65 g of Salvia miltiorrhiza, 40 g of Schisandra chinensis and 50 g of Ligustrum lucidum into crude particles, and performing thermal reflux extraction such as ethanol thermal reflux extraction on the crude particles twice;
(1) for the first time, adding 600 ml of ethanol into the crude particles for 3 h of reflux extraction to obtain a mixed solution; for the second time, adding 450 ml of ethanol into the mixed solution for 2 h of reflux extraction, and filtering to obtain filtrate; and
(2) decompressing the filtrate to recover ethanol to obtain extract;
Step S2: simultaneously placing 40 g of radix bupleuri, 40 g of Atractylodes macrocephala, 40 g of radix curcumae, 40 g of Angelica sinensis and 40 g of pericarpium citri reticulatae into a distillation vessel, adding 600 ml into the distillation vessel, and then di stilling to collect distillate;
Step S3: adding water into dregs obtained after reflux extraction of 65 g of astragalus membranaceus, 50 g of the fruit of Chinese wolfberry, 50 g of Tuckahoe, 40 g of Codonopsis pilosula, 40 g of Pinellia ternate, 40 g of rhizoma dioscoreae, 40 g of radix isatidis,40 g of Radix Paeoniae Alba, 40 g of Hedyotis diffusa, 40 g of honeysuckle, 40 g of rhizoma polygonati and 30 g of parched hawthorn fruit with ethanol and dregs obtained after extraction of distillate to be soaked for 1 h, and performing heating and extraction twice;
(1) for the first time, adding 5000 ml of water and decocting for 2 h to obtain a mixed solution; for the second time, adding 4000 ml of water and decocting for 1.5 h, and filtering to obtain a solution; and
(2) performing vacuum concentration on the solution, taking out the solution, adding a 5% 101 juice clarifying agent into the above solution, evenly stirring, standing and refrigerating for 24 h, and then performing vacuum concentration again to an appropriate amount to obtain a concentrated solution;
Step S4: adding the standby extract, the distillate and the concentrated solution, evenly stirring, adjusting a total volume to 1000 ml, packaging, sealing and sterilizing;
the external drug comprises 40 g of sedum sarmentosum, 40 g of barbed skullcap, 50 g of Hedyotis diffus, 10 g of Polygonum cuspidatum, 20 g of Fructus Aurantii, 30 g of Fructus Gardeniae, 40 g of oriental wormwood, 60 g of Schisandra chinensis and plaster cloth;
the decoction and extraction process of the external drug comprises the following steps:
Step S5: adding 1000 ml of water into 40 g of Sedum sarmentosum, 40 g of barbed skullcap, 50 g of Hedyotis diffusa, 10 g of Polygonum cuspidatum, 20 g of Fructus Aurantii, 30 g of Fructus Gardeniae, 40 g of oriental wormwood and 60 g of Schisandra chinensis to be soaked for 2 h;
Step S6: decocting the soaked medicinal materials for 15 min over fire to obtain a concentrated medicinal juice, and then filtering the concentrated medicinal juice to remove residues;
Step S7: pouring the filtered medicinal juice into a pot to be concentrated, decocting over high heat to accelerate the evaporation of water, and skimming off the foam at any time to gradually thicken the medicinal juice, then further concentrating over soft fire while continuously stirring until the medicinal juice does not scatter on paper because the medicinal juice is prone to sticking to the bottom, and stopping decocting at this moment to obtain a concentrated clear ointment;
Step S8: when the plaster is pasted, evenly smearing the clear ointment on plaster cloth, leaving a certain gap at the edge to facilitate the sealing of the edge, gently compacting the plaster cloth after pasting is completed to tightly bond the ointment with the plaster cloth.

2. The process for preparing an efficient liver-protecting compound ingredient according to claim 1, wherein in the step S3, an instrument used for vacuum concentration is a rotary evaporator.

3. The process for preparing an efficient liver-protecting compound ingredient according to claim 1, wherein the plaster cloth is cut into a size of 80 mm*80 mm, and the plaster cloth is made of a non-woven fabric material.

4. The process for preparing an efficient liver-protecting compound ingredient according to claim 1, wherein in the step S1, the concentration of ethanol is 80%, and the extraction temperature is 70° C.-75° C.

5. The process for preparing an efficient liver-protecting compound ingredient according to claim 1, wherein in the step S2, the distillation is short-range distillation, with a process pressure range of 1-1×10⁻³ mba and a temperature range of 150-280° C.

6. The process for preparing an efficient liver-protecting compound ingredient according to claim 1, wherein in the step S3, the ratio of vacuum concentration is 1:1.5, and an addition ratio of the 5% 101 fruit juice clarifying agent to the filtrate is 1:9.

7. The process for preparing an efficient liver-protecting compound ingredient according to claim 1, wherein in the step S6, filtration is performed by using a ceramic membrane filter, and the ceramic membrane filter is washed with a pH0-14 strong acid/alkaline oxidizing reagent.

8. The process for preparing an efficient liver-protecting compound ingredient according to claim 1, wherein in the step S1, the mesh size of the filter screen used for filtration is 1.20 mm, and the filter screen is made of a stainless steel material.
